(19) United States
(12) Patent Application Publication
(10) Pub. No.: US 2017/0166543 A1
(43) Pub. Date: Jun. 15, 2017

(54) COMPOSITIONS AND METHODS FOR SYNTHESIZING (2S,3S)-TRANS-EPOXYSUCCINYL-L-LEUCYL-AMIDO-3-METHYLBUTANE ETHYL ESTER

(71) Applicant: AMERICAN LIFE SCIENCE PHARMACEUTICALS, INC., San Diego, CA (US)

(72) Inventors: Robert J. Ternansky, San Diego, CA (US); Hui Liu, San Diego, CA (US); Emerich Eisenreich, Azusa, CA (US); Kenneth A. Haines, Azusa, CA (US); Daniel Levin, Azusa, CA (US); Xuejun Liu, Azusa, CA (US)

(21) Appl. No.: 15/115,893

(22) PCT Filed: Feb. 2, 2015
(86) PCT No.: PCT/US15/14131

§ 371 Date: Aug. 1, 2016

Related U.S. Application Data
(60) Provisional application No. 61/935,063, filed on Feb. 3, 2014.

(51) Int. Cl. C07D 301/00; C07B 59/00; C07C 231/12; C07C 237/06; C07C 209/26; C07C 269/06

(52) U.S. Cl. CPC C07D 301/00; C07C 209/26; C07C 269/06; C07C 231/12; C07C 237/06; C07B 59/002; C07B 2200/05

(57) ABSTRACT

In alternative embodiments the invention provides methods for synthesizing AB-007 (also called loxistatin, E64d, EST or ((2S,3S)-trans-epoxysuccinyl-L-leucyl-amido-3-methylbutane ethyl ester) and its acid form E64c (loxistatin acid), and various synthetic intermediates, and deuterated forms of these compounds, and stereoisomers thereof. In alternative embodiments the invention provides a tosylate salt of AB-007-4 or a tosylate salt of L-leucine isoamylamine, or equivalents thereof. A synthetic scheme of the invention provides kilogram quantities of AB-007 manufactured according to current good manufacturing practices (cGMP's), consistent with US FDA requirements for human use. In alternative embodiments the invention provides a tosylate salt of AB-007-4 or a tosylate salt of L-leucine isoamylamine, or equivalents thereof.

### GOVERNMENT RIGHTS

This invention was made with government support under grant number 4R44AG032784 awarded by the National Institutes of Health (NIH). The government has certain rights in the invention.

### FIELD OF THE TECHNOLOGY

The invention generally relates to synthetic and medicinal chemistry. In alternative embodiments the invention provides methods for synthesizing AB-007 (also called loxistatin, E64d, EST or ((2S,3S)-trans-epoxysuccinyl-L-leucyl-amido-3-methylbutane ethyl ester) and its acid form E64c (loxistatin acid), and various synthetic intermediates, and deuterated forms of these compounds, and stereoisomers thereof. In alternative embodiments the invention provides a tosylate salt of AB-007-4 or a tosylate salt of L-leucine isoamylamine, or equivalents thereof.

### BACKGROUND

AB-007 (also called loxistatin, E64d, EST or ((2S,3S)-trans-epoxysuccinyl-L-leucyl-amido-3-methylbutane ethyl ester) is an ethyl ester prodrug, 342.4 mol wt (MW), which is completely converted (hydrolyzed) in vivo to its acid form E64c (also called loxistatin acid or Ep 475, 314.4 mol wt, which irreversibly inhibits proteases belonging to the cysteine protease class by covalently binding to sulfhydryl groups in the proteases' active sites (Scheme A):

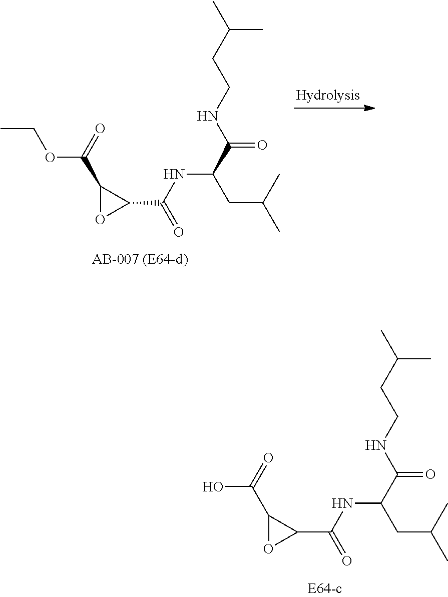 [SMILES: CCOC(=O)[C@@H]1O[C@H]1C(=O)N[C@H](CC(C)C)C(=O)NCCC(C)C ; CC(C)CCNC(=O)C(CC(C)C)NC(=O)C1OC1C(=O)O]

### SUMMARY

In alternative embodiments the invention provides methods for synthesizing AB-007 (also called loxistatin, E64d, EST or ((2S,3S)-trans-epoxysuccinyl-L-leucyl-amido-3-methylbutane ethyl ester), and various synthetic intermediates, and AB-007's acid form E64c (loxistatin acid), and deuterated forms of these compounds, such as 11-deuterated forms, and stereoisomers thereof. An exemplary synthetic scheme of the invention provides kilogram quantities of AB-007 manufactured according to current good manufacturing practices (cGMP's), consistent with US FDA requirements for human use.

In alternative embodiments the invention provides a tosylate salt of AB-007-4 or a tosylate salt of L-leucine isoamylamine, or equivalents thereof. In alternative embodiments the invention provides compositions comprising: a tosylate salt of AB-007-4; or, a tosylate salt of L-leucine isoamylamine; or, equivalents thereof.

In alternative embodiments the invention provides synthetic schemes (protocols, routes or methods) comprising at least one protocol/scheme: (a) as set forth in FIG. 1; (b) as set forth in Scheme Ia, Scheme Ib, Scheme II, Scheme C and/or Scheme D, of Example I; or (c) equivalents of (a) and (b).

In alternative embodiments the invention provides synthetic schemes (protocols) for making AB-007, comprising:

(a) Step 1 of the following Scheme, and equivalents thereof:

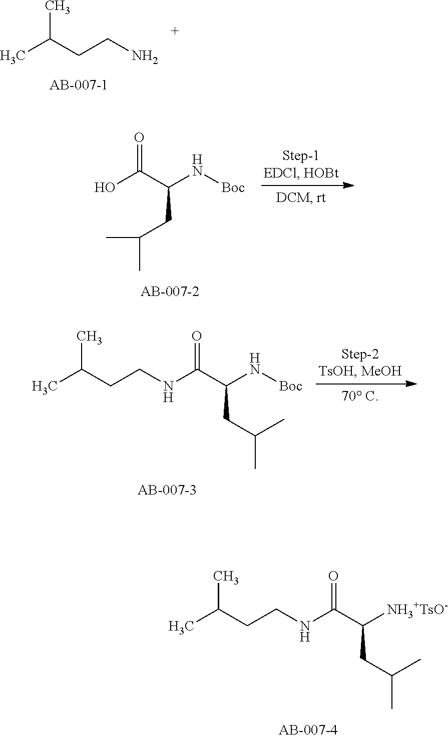

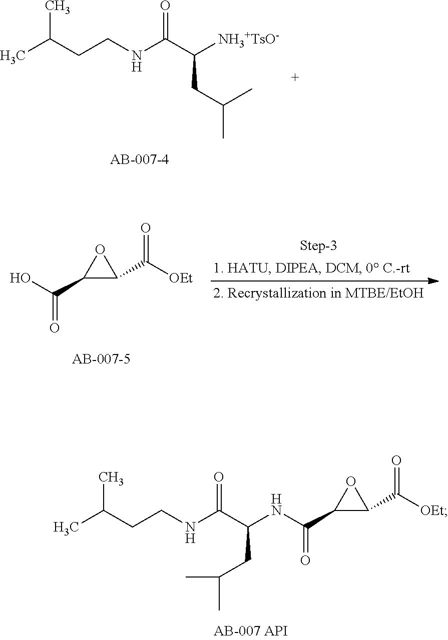

or

(b) the synthetic scheme of (a), wherein the AB-007 has one, two or all three of the three chiral centers of E64c.

In alternative embodiments the invention provides synthetic schemes (protocols, routes or methods) comprising Step 2 of the following Scheme, and equivalents thereof:

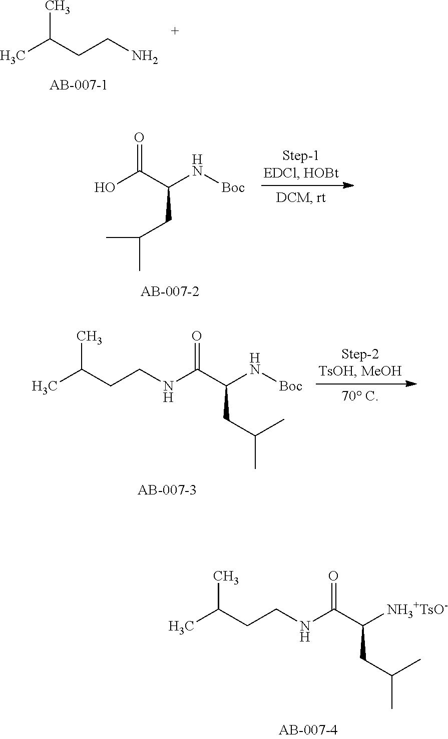

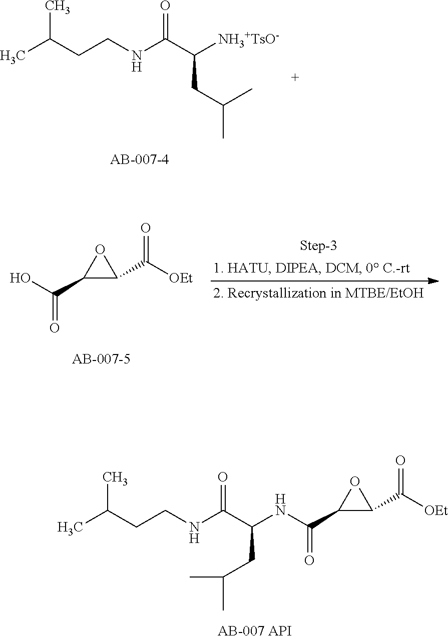

In alternative embodiments the invention provides synthetic schemes (protocols, routes or methods) comprising Step 3 of the following Scheme, and equivalents thereof:

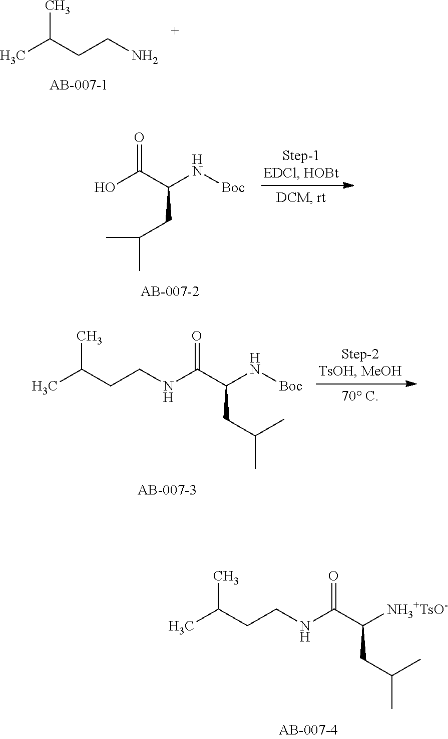

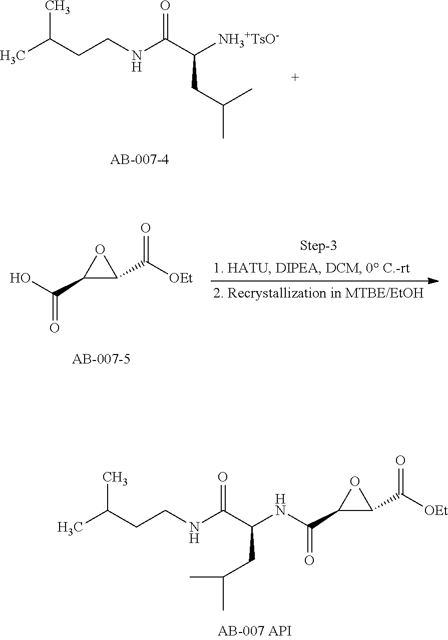

In alternative embodiments the invention provides synthetic schemes (protocols, routes or methods) comprising Step 1 and Step 2 of the following Scheme, and equivalents thereof:

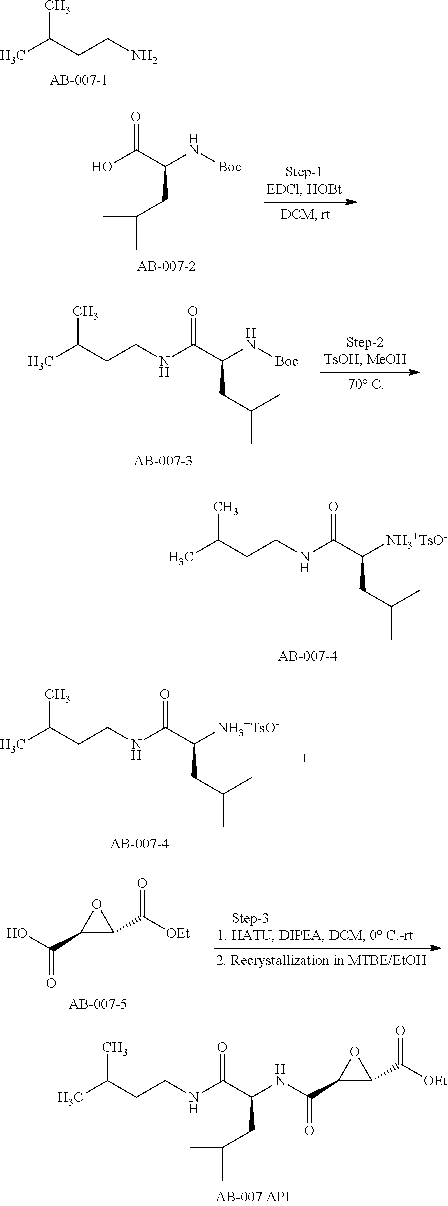

In alternative embodiments the invention provides synthetic schemes (protocols, routes or methods) comprising Step 2 and Step 3 of the following Scheme, and equivalents thereof:

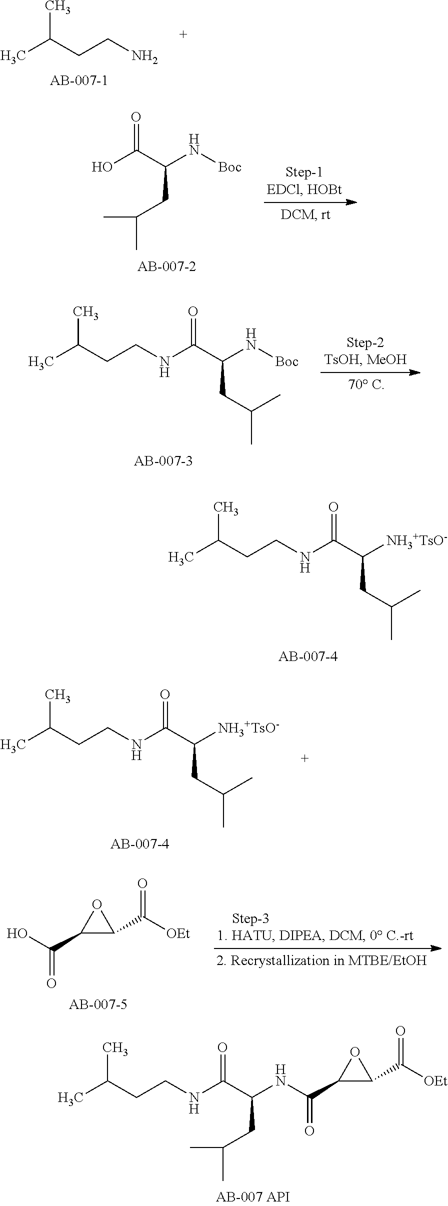

In alternative embodiments the invention provides synthetic schemes (protocols, routes or methods) comprising the synthetic scheme of the invention (e.g., as described above), wherein:

(a) in place of using EDCI/HOBt standard peptide coupling conditions for a coupling reaction (e.g., optionally, as in Scheme I), a reagent comprising or consisting of propylphosphonic anhydride, or propane phosphonic acid anhydride (T3P®), is used for the coupling reaction;

(b) in place of using BOC deprotection of AB-007-2 starting material (e.g., optionally, before step 1 of Scheme I), alternative conditions comprising controlled addition of a solution of AB-007-3 into a warm solution of excess p-toluene sulfonic acid can be used to provide effective control of gas evolution rate by adjusting the addition rate of the AB-007-3 solution, as needed; these conditions can be used to ensure a good control of the rate of carbon dioxide gas evolution throughout the course of the reaction, along with isolation of the Step 2 product as a “free base” form to avoid toluene sulfonic acid carry over to the subsequent coupling step;

(c) drying of a hydrated AB-007-2 starting material (e.g., optionally, before step 1 of Scheme I), and optionally removal of water of a hydrated AB-007-2 by azeotropic drying;

(d) isolating a solid AB-007-3 (e.g., optionally, after step 1 of Scheme I) by crystallization, and optionally by crystallization from water and acetone.

(e) for a BOC deprotection step (e.g., optionally, step 2 of Scheme I), controlling addition of an AB-007-3 solution into an excess warm acid solution for control of gas evolution through addition control;

(f) isolating AB-007-4 (e.g., optionally, after step 2 of Scheme I) as a free base form to avoid issues with carry-over of residual p-TSA;

(g) exchanging solvent to ethyl acetate in step 3, Scheme I, to enable removal of TMU (or 1,1,3,3-tetramethylurea) by one or more aqueous washes;

(h) controlling bis-amide by-product in step 3, Scheme I, using an approach comprising:
(1) using an alkaline metal salt form to limit degradation of the acid form of AB-007-5, which can be unstable, causing disproportionates forming a reactive di-acid form;
(2) using saturated brine wash of an organic solution of AB-007-5 in its acid form to remove traces of di-acid; and/or
(3) removing a bis-amide impurity by filtration of the reaction mixture;

(i) in place of needing chromatography, two critical process impurities are controlled by process conditions comprising: for the control of tetramethylurea (TMU), after a solvent exchange to ethylacetate, an organic solution is washed with 6.5% (w/w) sodium bicarbonate solution, de-ionized water, and 1 M monosodium phosphate, and de-ionized water;

(j) in place of needing chromatography by solvent exchange and aqueous washing of reaction product solution to remove impurities (a by-product of coupling chemistry), AB-007-5 raw material (or AB-007-5 made by step 3, Scheme Ia) is prepared in a neutral-pH sodium salt form instead of a free acid form so as to avoid residual acidity causing its disproportionation (bis-acid impurity present in AB-007-5 is due to disproportionation of AB-007-5 into (reactive) bis-acid and (unreactive) bis-ester, catalyzed by residual mineral acid present in AB-007-5);

(k) the method of (j), wherein a brine wash also is employed to remove small amounts of remaining bis-acid in AB-007-5; and (based on solubility studies which showed low solubility of the unwanted bis-amide), the optionally, small amount of bis-amide formed in the final step coupling is removed by filtration and AB-007 API recrystallization;

(l) the amount of bis-amide by-product can be controlled by a protocol comprising:
(1) using an alkaline metal salt form to limit degradation (the acid form of AB-007-5 is unstable and disproportionates form reactive di-acid forms), and/or
(2) using a saturated brine wash of an organic solution of AB-007-5 in its acid form to remove traces of di-acid, and/or
(3) removing by filtration bis-amide impurities (which can have a surprisingly low solubility in the reaction mixture prior to further work up and can be removed by filtration of the reaction mixture); or

(l) any combination of, or at least one of, (a) to (k), or all of (a) to (k).

In alternative embodiments the invention provides synthetic schemes (protocols, routes or methods) for making isoamylamine-d11 (and equivalents), or for making 11-deuterated AB-007 (or E64d) comprising the following steps, or equivalents:

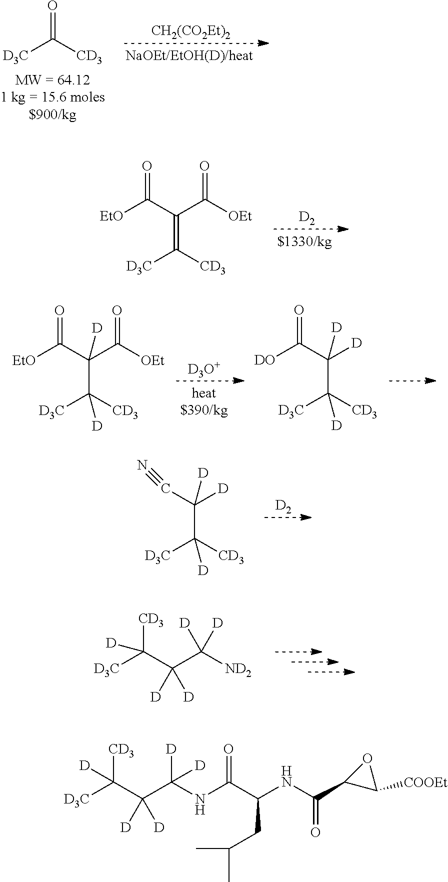

Estimated yield of isoamylamine-d11(13) on kg scale: 664 g of isoamylamine-d11(13) gives a projected amt of about 930 g E64D-d11
(5.11 moles amine synthesized if 80% yield is obtained on each of the five steps)
Estimated cost on kg scale of reagents: $2620+non-deuterated reagents/solvents
Comparison of deuterated vs. non-deuterated routes:
cost of d11 isoamylamine: $2620/kg
# steps to synthesize d11 isoamylamine: 5 (adds additional manufacturing costs as well)
cost of non-deuterated isoamyl amine: $82/kg (from Aldrich)
# steps to synthesize non-deuterated isoamylamine: Zero

In alternative embodiments the invention provides synthetic schemes (protocols, routes or methods) for synthesizing AB-007-3-(eleven deuterated, or d-11), or equivalents, comprising:

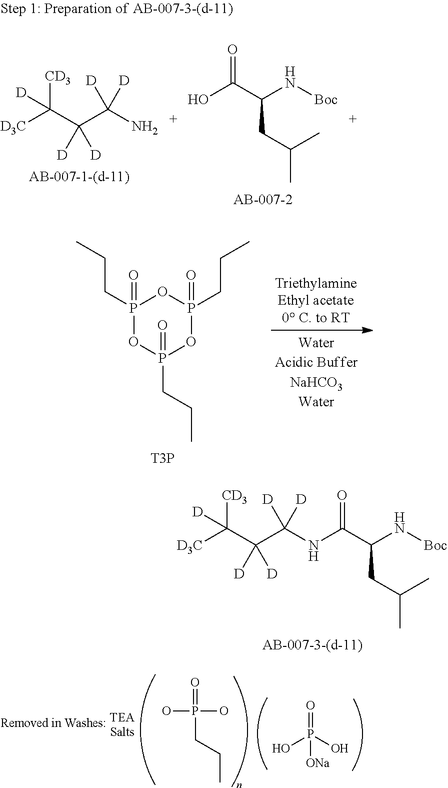

In alternative embodiments the invention provides synthetic schemes (protocols, routes or methods) for synthesizing AB-007-4-(eleven deuterated, or d-11), or equivalents, comprising:

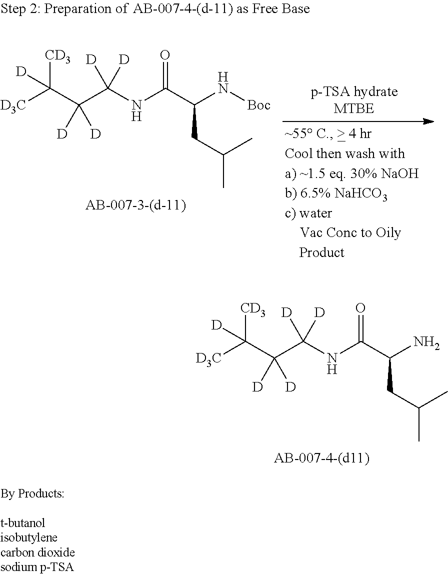 [SMILES: [2H]C([2H])([2H])C([2H])(C([2H])([2H])[2H])C([2H])([2H])C([2H])([2H])NC(=O)[C@H](CC(C)C)NC(=O)OC(C)(C)C ; [2H]C([2H])([2H])C([2H])(C([2H])([2H])[2H])C([2H])([2H])C([2H])([2H])NC(=O)[C@@H](N)CC(C)C]

In alternative embodiments the invention provides synthetic schemes (protocols, routes or methods) for synthesizing AB-007-(eleven deuterated, or d-11), from AB-007-4-(eleven deuterated, or d-11), or equivalents, comprising:

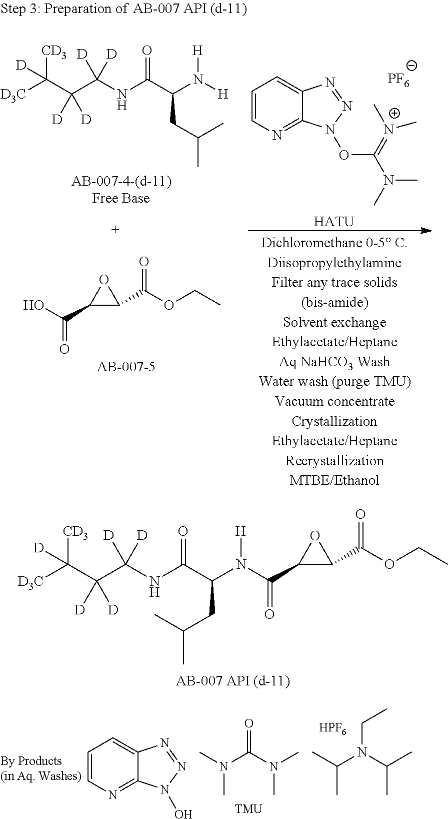

All publications, databases, patents, and patent applications cited in this specification are herein expressly incorporated by reference as if each was specifically and individually indicated to be incorporated by reference.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 graphically illustrates an exemplary synthetic scheme of the invention.

Like reference symbols in the various drawings indicate like elements.

### DETAILED DESCRIPTION

In alternative embodiments the invention provides methods for synthesizing AB-007 (E64d, or loxistatin) and its acid form E64c (loxistatin acid), and deuterated forms of these compounds, and stereoisomers thereof, for example, comprising the three chiral centers of E64c. One exemplary synthetic scheme of the invention provides kilogram quantities of AB-007 manufactured according to current good manufacturing practices (cGMP's), consistent with US FDA requirements for human use.

In alternative embodiments the invention provides a tosylate salt of AB-007-4 or a tosylate salt of L-leucine isoamylamine, or equivalents thereof.

The following examples are provided to further illustrate the invention but not to limit its scope. Other variants of the invention will be readily apparent to one of ordinary skill in the art.

### EXAMPLES

Standard procedures and chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-83, John Wiley and Sons, New York, N.Y., 2006; March J. and Smith M., Advanced Organic Chemistry, 6th ed., John Wiley and Sons, New York, N.Y.; and Larock R. C., Comprehensive Organic Transformations, Wiley-VCH Publishers, New York, 1999. All texts and references, patents and patent applications cited herein are expressly incorporated by reference in their entirety.

Reactions using compounds having functional groups may be performed on compounds with functional groups that may be protected. A “protected” compound or derivatives means derivatives of a compound where one or more reactive site or sites or functional groups are blocked with protecting groups. Protected derivatives are useful in the preparation of the compounds of the present invention or in themselves; the protected derivatives may be the biologically active agent. Examples suitable protecting groups that can be used to practice this invention can be found in e.g., T. W. Greene, Protecting Groups in Organic Synthesis, 3rd edition, John Wiley & Sons, Inc. 1999; or T. W. Greene and P. G. M. Wuts, Protecting Groups in Organic Synthesis, 4th edition, John Wiley & Sons, Inc. 2007.

### Example 1—Exemplary Protocols to Synthesize E64d and E64c

In alternative embodiments, the invention provides protocols comprising Scheme Ia Scheme Ib, and Scheme II, including routine variations thereof.

### Scheme Ia

One exemplary embodiment, AB-007 is synthesized by the following Scheme Ia:

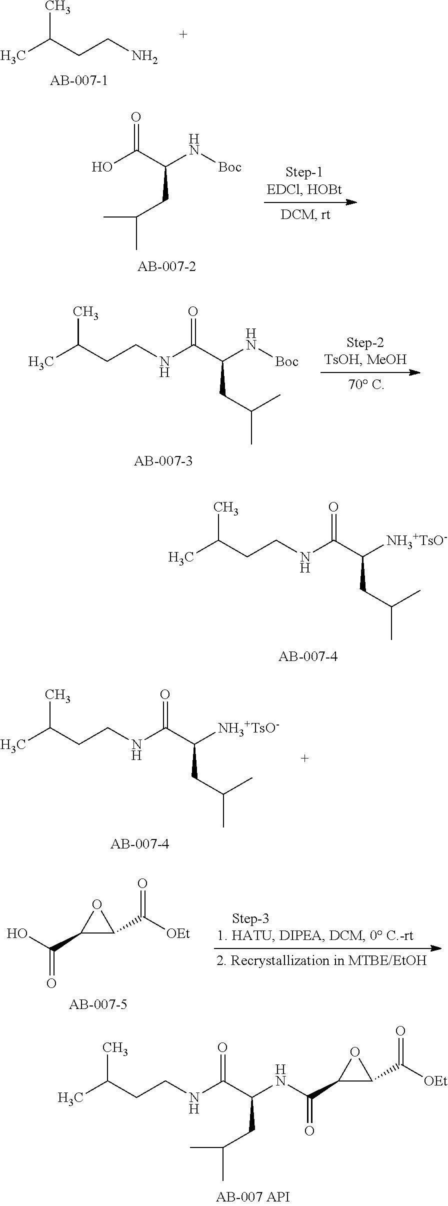

### Synthetic Route Overview

A synthetic route for E64d as described by Tashio is schematically outlined below as Scheme B. This route involved preparation of p-nitrophenol active ester 6, which was coupled with free L-leucine isoamylamine (3) to provide E64d (see e.g., Tamai, M., Yokoo, C., Murata, M., Oguma, K., Sota, K., Sato, E., Kanaoka, Y. Chem. Pharm. Bull. 1987, 35, 1098). In this route, the key intermediate 6 and E64d were prepared only in modest yields. Also, L-leucine isoamylamine was used as an oily crude product without proper purification and characterization. In order to overcome the drawbacks of this original route, this invention provides a new process to synthesize E64d.

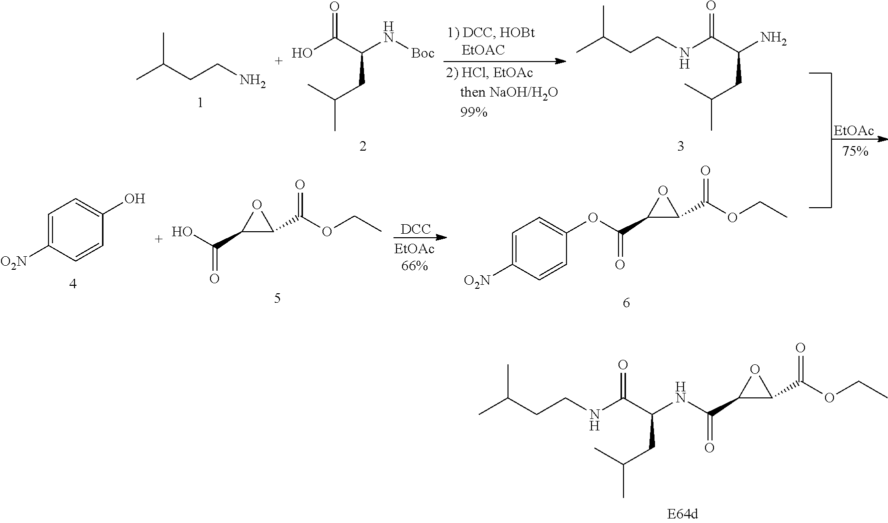

Scheme B: Original Synthetic Route as Described by Tashio

In the new, exemplary approach of this invention, the synthesis of E64d (Scheme 2) was initiated by coupling Boc-Leu (Boc is tert-butoxycarbonyl) monohydrate (2) with isoamylamine (1) in the presence of EDCI (1-ethyl-3-3-dimethylaminopropyl)carbodiimide) and HOBt (N-hydroxy-benzotriazole) monohydrate. Boc deprotection of L-leucine isoamylamine 3a with p-toluenesulfonic acid monohydrate in methanol afforded the intermediate 3b as a tosylic salt. Intermediate 3b was then treated with epoxy acid 5 in the presence of HATU (a peptide coupling reagent, 2-(7-Aza-1H-benzotriazole-1-yl)-1,1,3,3-tetramethyluronium hexafluorophosphate) and diisopropylethylamine to provide E64d in almost quantitative yield.

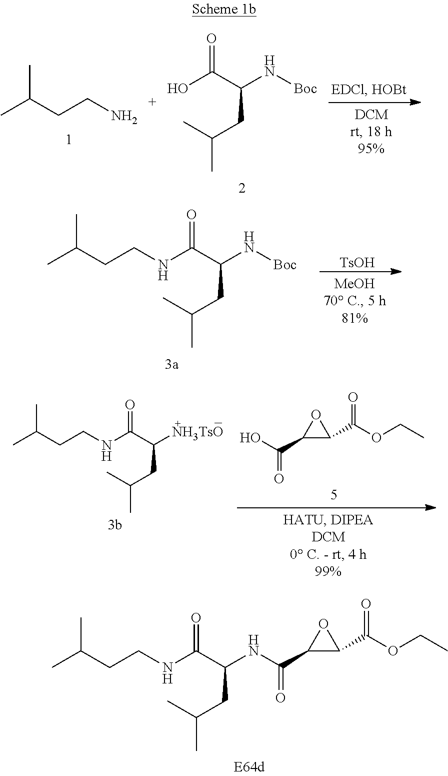

### Synthesis of Boc-L-Leucine Isoamylamine 3a

Boc-L-leucine isoamylamine 3a was obtained in high yield using standard coupling methodology. Boc-Leu monohydrate was coupled with isoamylamine in the presence of EDCI and HOBt at room temperature to furnish the required amide product in over 90% yield after purification.

### Boc Deprotection and Salt Formation

The Boc deprotection in the original route was carried out with a solution of HCl in ethyl acetate to provide hydrochloride salt of L-leucine isoamylamine (3c) (Scheme C). However, it was found that hydrochloride salt 3c was very difficult to handle in air due to its highly hydroscopic nature. Considering that this material is a GMP intermediate which has to be prepared in large scale, it was necessary to find out a more appropriate salt form of L-leucine isoamylamine. Carboxylic acid salts should be avoided in this case because byproducts could form in the following amide bond formation reaction (Scheme 2). An acid which could remove the Boc protecting group and also provide a well-behaved salt simultaneously would be an ideal choice. Several acids were tested in order to find the suitable conditions for Boc deprotection and salt formation. The results of these experiments are summarized in Table 1. Among the acids tested, p-toluenesulfonic acid seemed to give the best salt form. The tosic acid salt in non-hydroscopic and easy to handle.

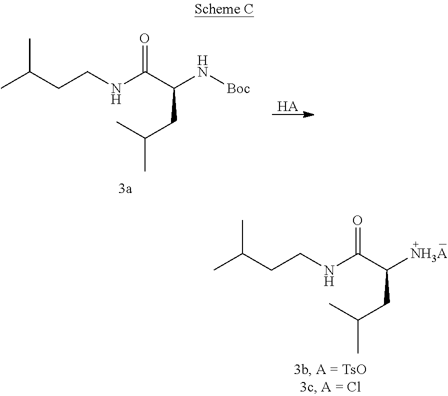

TABLE 1

| Entry | Acid tested | Solvents for salt formation | Salt property |
|---|---|---|---|
| 1 | HCl | N/A, purchased from CRO | White solid, |
| | | | very hydroscopic |
| 2 | HBr | diethyl ether/hexanes | oil |
| 3 | H₂SO₄ | diethyl ether/hexanes | oil |
| 4 | p-TsOH | diethyl ether/hexanes/methanol | White crystal, |
| | | | mp 114-115° C. |
| 5 | TFA | diethyl ether/hexanes | oil |

### Preparation of E64d

Commonly used peptide coupling reagents, such as EDCI/HOBt, mixed anhydride (prepared from isopropyl chloroformate), PyBop (benzotriazol-1-yl-oxytripyrrolidinophosphonium hexafluorophosphate), CDI (carbonyl diimidazole), HATU, HBTU (O-Benzotriazole-N,N,N′,N′-tetramethyl-uronium-hexafluoro-phosphate), and HCTU (2-(6-Chloro-1H-benzotriazole-1-yl)-1,1,3,3-tetramethylaminium hexafluorophosphate), were used in the preparation of E64d. Based on the LC-MS (liquid chromatography-mass spectrometry) analysis results of crude reaction mixtures, it was found that the coupling method using HATU provided E64d with the highest crude purity and no byproduct formation. E64d was easily obtained in almost quantitative yield after aqueous workup and purification via a silica gel plug eluting with 50% EtOAc in hexanes. The results of the coupling experiments are summarized in Table 2.

Scale-up of the synthesis of E64d utilized the hydrochloride salt of L-leucine isoamylamine (3c; Scheme C) in the presence of HATU. This provided over 50 g of E64d. The yield and purity of final product were consistent with the results obtained using the p-toluenesulfonic acid salt of L-leucine isoamylamine (3b).

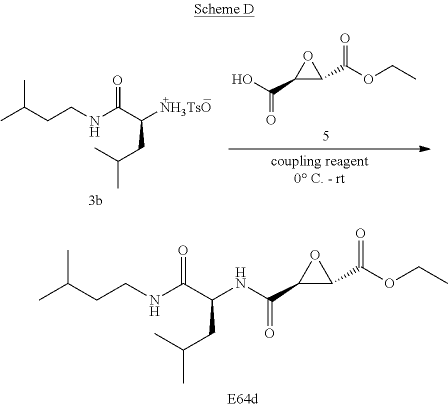

TABLE 2

| | Coupling | | | | |
|---|---|---|---|---|---|
| Entry | reagent | Solvent | Temp | Time | Result after standard process* |
| 1 | PyBop | CH₂Cl₂ | 0° C.-rt | 0° C., 0.5 h; | Product obtained in high yield (>95%) |
| | | | | rt, 42 h | with some impurities |
| 2 | CDI | CH₂Cl₂ | rt | 42 h | Product obtained in low yield and |
| | | | | | low purity |
| 3 | HATU | CH₂Cl₂ or | 0° C.-rt | 0° C., 0.5 h; | Product obtained in high yield (>95%) |
| | | MTBE/DMF or | | rt, 42 h | and high purity (>98% by |
| | | EtOAc/DMF | | | UV215 nm and ELSD) |
| 4 | HBTU | CH₂Cl₂ | rt | rt, 115 h | Product obtained in high yield (>95%) |
| | | | | | with some impurities |
| 5 | HCTU | CH₂Cl₂ | rt | rt, 115 h | Product obtained in modest yield |
| | | | | | (75%) with some impurities |
| 6 | EDCI/HOBt | CH₂Cl₂ | 0° C.-rt | 0° C. 1 h, | Product obtained in low yield and |
| | | | | rt 90 h | low purity |
| 7 | Isopropyl | MTBE/THF | 0° C.-rt | 0° C., 0.5 h; | Low conversion, an unknown by- |
| | chloroformate | | | rt, 24 h | product formed as major product |

*Reaction was carried out on 1 mmol scale. The reaction mixture was worked up and purified by a short silica gel plug (1 cm D × 6 cm H) eluting with 50% EtOAc in hexanes.

### Polymorphism Study of E64d

In order to evaluate its crystal forms, E64d was recrystallized in several solvent systems, including diisopropylether/ethanol, diisopropylether/ethyl acetate, t-butylmethylether/ethanol, isopropanol, ethyl acetate/hexanes, toluene/hexanes, and ethanol/water. The results of recrystallization are summarized in Table 3.

TABLE 3

| Entry | Solvents | Yield (%) | mp of crystal (° C.) |
|---|---|---|---|
| 1 | diisopropylether/ethanol | 86 | 123-124 |
| 2 | diisopropylether/ethyl acetate | 87 | 123-124 |
| 3 | t-butylmethylether/ethanol | 76 | 123-124 |
| 4 | isopropanol | 60 | 124-125 |
| 5 | ethyl acetate/hexanes | 93 | 123-124 |
| 6 | toluene/hexanes | 94 | 123-124 |
| 7 | ethanol/water | 60 | 123-124 |

All reagents were commercial grade and were used as received without further purification, unless otherwise specified. Commercially available anhydrous solvents were used for reactions conducted under inert atmosphere. ¹H- and ¹³C-NMR spectra were recorded at 500 and 125 MHz, respectively. Chemical shifts for ¹H-NMR spectra are given in parts per million with respect to tetramethylsilane (0.00 ppm), which was used as a reference. ¹³C-NMR spectra were referenced with respect to NMR solvent (CDCl₃, 77.1 ppm; DMSO-d₆, 39.5 ppm). Mass spectra (MS) were recorded on a PE/Sciex API-150EX mass spectrometer using APCI. The purity of compounds were analyzed on an HPLC equipped with a Waters 1525™ pump, a Waters 2487™ dual UV/Vis Absorbance detector, a Waters 717 Plus™ autosampler and a Phenomenex Luna C18™ (2) 100 mm×4.6 mm reverse-phase column using a gradient with solvents [A] 0.1% trifluoroacetic acid in water and [B] 0.1% trifluoroacetic acid in acetonitrile with a flow rate of 1 mL/min.

### Intermediate Synthesis

### (S)-t-Butyl 1-(isopentylamino)-4-methyl-1-oxopentan-2-ylcarbamate (3a) (HLI-014-008)

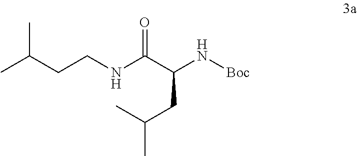 [SMILES: CC(C)CCNC(=O)[C@H](CC(C)C)NC(=O)OC(C)(C)C]

To a solution of Boc-L-Leu.H₂O (5.0 g, 22 mmol) in CH₂Cl₂ (200 mL) were added 1-hydroxybenzotriazole hydrate (4.3 g, 28 mmol) and isoamylamine (2.0 g, 23 mmol), followed by 1-ethyl-3-(3-dimethylaminopropyl)carbodiimide hydrochloride (6.2 g, 32 mmol) at room temperature under nitrogen. The reaction mixture was stirred at room temperature for 18 h and then diluted with CH₂Cl₂ (150 mL). The resulting solution was washed with saturated sodium bicarbonate solution (2×) and saturated sodium chloride solution (1×), dried over Na₂SO₄, filtered, and concentrated under reduced pressure to afford product 3a as a white foam (6.2 g, 95%): APCI MS m/z 301.4 (M+H)⁺. This material was used for the next step without further purification.

### (S)-1-(Isopentylamino)-4-methyl-1-oxopentan-2-aminium 4-methylbenzenesulfonate (3b) (HLI-014-029)

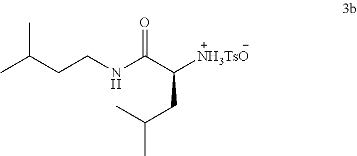

To a solution of (S)-t-butyl 1-(isopentylamino)-4-methyl-1-oxopentan-2-ylcarbamate (3a) (4.1 g, 14 mmol) in methanol (30 mL) was added p-toluenesulfonic acid hydrate (3.3 g, 17 mmol) at room temperature. The reaction mixture was stirred at 70° C. for 5 h. After the reaction mixture was cooled to room temperature, the solvent was removed under vacuum to afford an oily residue, which was triturated in a mixture of diethyl ether and hexanes to provide compound 3b as a white foam (5.1 g). The white foam was dissolved in 20% EtOAc in hexanes (100 mL) at 80° C. and then cooled to about 40° C. To this warm solution was added crystal seed (20 mg) and allowed the mixture to cool to room temperature. The crystal that formed was collected by filtration, dried in vacuo to afford product 3b as white needles (4.1 g, 81%): mp 114-115° C.; ¹H NMR (500 MHz, DMSO-d₆) δ8.43 (t, J=5.4 Hz, 1H), 8.08 (br s, 3H), 7.50 (d, J=7.9 Hz, 2H), 7.12 (d, J=7.9 Hz, 2H), 3.68-3.69 (m, 1H), 3.13-3.19 (m, 1H), 3.04-3.08 (m, 1H), 2.29 (s, 3H), 1.55-1.62 (m, 2H), 1.50-1.54 (m, 2H), 1.28-1.32 (m, 2H), 0.85-0.89 (m, 12H); ¹³C NMR (125 MHz, DMSO-d₆) ε 168.5, 145.4, 137.8, 128.1, 125.5, 51.1, 40.2, 37.7, 36.9, 25.0, 23.7, 22.4, 22.3, 22.2, 22.1, 20.8; APCI MS m/z 201.4 (aminium C₁₁H₂₅N₂O⁺).

### Synthesis of E64d

### (2S,3S)-Ethyl-3-((S)-1-(isopentylamino)-4-methyl-1-oxopentan-2-ylcarbamoyl)oxirane-2-carboxylate (E64d) (ALP-466.000.13, HLI-014-027, HLI-014-036)

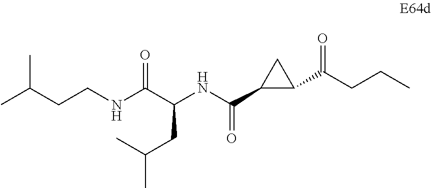 [SMILES: CCCC(=O)[C@H]1C[C@@H]1C(=O)N[C@@H](CC(C)C)C(=O)NCCC(C)C]

### Example 1 (ALP-466.000.13, HLI-014-027, HLI-014-036)

To a solution of (2S,3S)-3-(ethoxycarbonyl)oxirane-2-carboxylic acid (5) (4.6 g, 27 mmol) and (S)-1-(isopentylamino)-4-methyl-1-oxopentan-2-aminium 4-methylbenzenesulfonate (3b) (11 g, 29 mmol) in CH₂Cl₂ (150 mL) at 0° C. (ice-water bath) under a nitrogen atmosphere was added 2-(7-azabenzotriazol-1-yl)-1,1,3,3-tetramethyluronium hexafluorophosphate (12 g, 32 mmol), followed by diisopropylethylamine (10 mL, 57 mmol). After stirring at 0° C. for 1.5 h, the ice bath was removed and the mixture was stirred at room temperature for 2.5 h. The mixture was diluted with CH₂Cl₂ (300 mL) and then washed successively with saturated sodium bicarbonate solution (2×) and saturated sodium chloride solution (1×), dried over Na₂SO₄, filtered and concentrated in vacuo to afford the crude product as a yellow viscous oil. The crude product was filtered through a silica gel plug (70 mm×110 mm D×H) eluting with 10% of ethyl acetate in hexanes followed by 50% of ethyl acetate in hexanes. Concentration of the 50% of ethyl acetate in hexanes elution fractions provided E64d (9.7 g, 99%) as a white solid: mp 122-123° C. Recrystallization in MTBE-EtOH afforded E64d as white short needles (80%): mp 123-124° C.; ¹H NMR (500 MHz, CDCl₃) ε 6.71 (d, J=8.4 Hz, 1H), 6.13 (br s, 1H), 4.37-4.41 (m, 1H), 4.21-4.28 (m, 2H), 3.67 (d, J=1.9 Hz, 1H), 3.46 (d, J=1.9 Hz, 1H), 3.19-3.30 (m, 2H), 1.49-1.65 (m, 4H), 1.38 (q, J=7.4 Hz, 2H), 1.30 (t, J=7.0 Hz, 3H), 0.89-0.93 (m, 12H); APCI MS m/z 343.2 (M+H)⁺; Anal. (C₁₇H₃₀N₂O₅) C, H, N; HPLC purity: 99.6%.

### Example 2 (HLI-014-025, HLI-014-035)

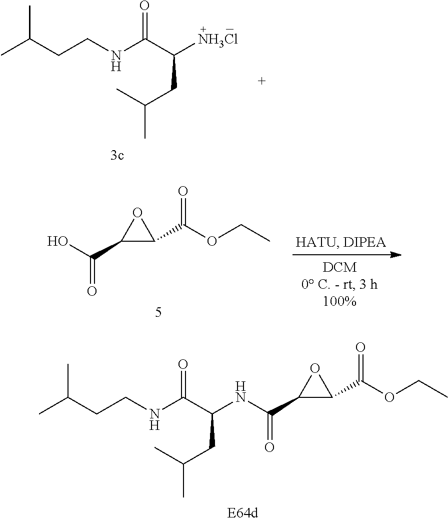

To a solution of (2S,3S)-3-(ethoxycarbonyl)oxirane-2-carboxylic acid (5) (25 g, 0.16 mol) and (S)-1-(isopentylamino)-2-amino-4-methyl-1-oxopentan hydrochloride (3c) (38 g, 0.16 mol) in CH₂Cl₂ (750 mL) at 0° C. (ice-water bath) under a nitrogen atmosphere was added 2-(7-azabenzotriazol-1-yl)-1,1,3,3-tetramethyluronium hexafluorophosphate (65 g, 0.17 mol), followed by diisopropylethylamine (DIPEA, N,N-diisopropylethylamine) (56 mL, 0.31 mol). After stirring at 0° C. for 1 h, the ice bath was removed and the mixture was stirred at room temperature for 2 h. The mixture was diluted with CH₂Cl₂ (750 mL) and then washed successively with saturated sodium bicarbonate solution (2×) and saturated sodium chloride solution (1×), dried over Na₂SO₄, filtered and concentrated in vacuo to afford the crude product as a yellow viscous oil. The crude product was filtered through a silica gel plug (60 mm×300 mm D×H) eluting with 10% to 50% of ethyl acetate in hexanes. Concentration of the combined elution fractions (2.5 L) provided E64d (53 g, 100%) as a white solid. Recrystallization (36 g) in 1% EtOH in MTBE (methyl tert-butyl ether) (535 mL) afforded E64d as white short needles (25 g, 69%): mp 123-124° C.; ¹H NMR (500 MHz, CDCl₃) δ6.68 (d, J=8.4 Hz, 1H), 6.08 (br s, 1H), 4.36-4.40 (m, 1H), 4.21-4.28 (m, 2H), 3.67 (d, J=1.9 Hz, 1H), 3.46 (d, J=1.9 Hz, 1H), 3.19-3.30 (m, 2H), 1.49-1.65 (m, 4H), 1.38 (q, J=7.4 Hz, 2H), 1.30 (t, J=7.0 Hz, 3H), 0.89-0.93 (m, 12H); ESI MS m/z 343.2 (M+H)⁺; Anal. (C₁₇H₃₀N₂O₅) C, H, N; HPLC purity: 99.6%.

### Example II: Alternative Exemplary Protocols to Synthesize E64d and E64c

Alternative exemplary synthetic schemes of the invention include the exemplary Scheme Ia, Scheme Ib and Scheme II embodiments and variations and combinations thereof. The exemplary Scheme II can comprise one, several or all of the following variations of Scheme Ia or Scheme Ib:

In the Step 1 coupling chemistry, an alternative coupling reagent was designed in place of the EDCI/HOBt standard peptide coupling conditions—with a view to avoiding costly preparative chromatography necessary to remove process impurities with EDCI/HOBt coupling conditions. Propylphosphonic anhydride, or propane phosphonic acid anhydride, (T3P®, Archimica Inc., Origgio, Italy/Wilmington, Del.) was identified as a more efficient coupling reagent for this step which, along with the other process improvements to Scheme Ia as described below, allowed synthesis of the Step 1 product in over 90% yield and in over 98% purity without the need for preparative chromatography.

In summary, for the alternative, exemplary embodiments of Step 1 of Scheme Ia:
a. The use of T3P for the coupling reaction provides a very clean process stream and avoids the impurities seen with other coupling reagents, mitigating the need for purification by chromatography and enabling the isolation of AB-007-3 as a filterable solid.
b. Removal of water by azeotropic drying of the AB-007-2, or drying of AB-007-2 by some other means, is critical to obtaining good yield and purity. The material is only commercially available in the hydrated form.
c. The isolation of solid AB-007-3 in good yield and purity by crystallization from water and acetone.

In Scheme Ia the step 2 (second step), or in Scheme Ib, which comprise BOC deprotection, gives rise to carbon dioxide as a gaseous by-product which, if not well controlled, can result in pressurization and explosion hazard on scale up. Although in some embodiments Scheme Ia or Scheme Ib conditions are suitable for small scale preparation, they do not give control of carbon dioxide evolution rate. Scheme Ia or Scheme Ib conditions also rely on evaporation to dryness for solid isolation; and can leave variable amounts of residual toluene sulfonic acid present in the Step 2 product (isolated as a toluene sulfonate salt) which may cause difficulties for the subsequent coupling step.

To address these issues, alternative exemplary conditions can be used to ensure, e.g., good control of the rate of carbon dioxide gas evolution throughout the course of the reaction, along with isolation of the Step 2 product as a “free base” form to avoid toluene sulfonic acid carry over to the subsequent coupling step: for example, in one embodiment, the safe gas evolution rate is unique to each reactor system and must be calculated separately for each case. In another embodiment, controlled addition of a solution of AB-007-3 into a warm solution of excess p-toluene sulfonic acid provides effective control of gas evolution rate by adjusting the addition rate of the AB-007-3 solution, as needed.

In one embodiment, for Scheme Ia or Scheme Ib, conditions for the final step of coupling AB-007-4 with AB-007-5 to give the AB-007 API requires preparative chromatography to isolate the API free from some significant process impurities.

In alternative embodiments, using chromatography as a final preparative step for the preparation of material for clinical trials may introduce a steep ongoing cost burden for alternative, e.g., scaled up, manufacture of AB-007. This may be because chromatography removes related substances, giving (resulting in) a very pure API. If this very pure API is used to qualify through toxicology testing and clinical trials (e.g., if the very pure API is used in toxicology testing for FDA approval for clinical trials), it would make it very difficult to avoid using (not also use) chromatography in alternative, e.g., scaled up, protocols. Not using chromatography (or an alternative apparatus or protocol yielding a high quality purification) in alternative, e.g., scaled up, protocols would result in increased impurity levels; and these impurity levels would then exceed qualified (low impurity) levels present in the chromatographed material, and thus would not be acceptable to the FDA.

In Scheme Ia or Scheme Ib, two major impurities were identified:
(i) tetramethylurea by-product of coupling chemistry, and
(ii) symmetrical bis-amide formed from reaction of AB-007-4 with bis-acid impurity present in AB-007-5.

In the process of Scheme II, tetramethylurea was successfully removed without need for chromatography by solvent exchange and aqueous washing of reaction product solution. It was found that bis-acid impurity present in AB-007-5 is due to disproportionation of AB-007-5 into (reactive) bis-acid and (unreactive) bis-ester, catalyzed by residual mineral acid present in AB-007-5. To address this, AB-007-5 raw material is prepared in: neutral-pH sodium salt form, or as an alkaline metal salt (optionally a sodium or potassium salt form), instead of the free acid form, e.g., to avoid residual acidity causing its disproportionation.

In one embodiment, a brine wash also is employed to remove the small amount of remaining bis-acid from the organic solvent solution of AB-007-5 in acid form. Based on solubility studies which showed a low solubility (a surprisingly low solubility) of the unwanted bis-amide in the reaction mixture before further work up, the small amount of bis-amide formed in the final step coupling was removed by filtration of the reaction mixture prior to the quench and work up.

In these alternative embodiments, process changes allowed definition of a robustly scaleable and efficient manufacturing process giving high purity AB-007 API without need for expensive preparative chromatography. For example, an exemplary process has produced as much as 1.096 kg of AB-007 suitable for clinical trials.

In summary, for the alternative, exemplary embodiments of Step 2:
a. Controlled addition of the AB-007-3 solution into excess warm acid solution for control of gas evolution through addition control.
b. Isolation of the AB-007-4 as the free base form to avoid issues with carry-over of residual p-TSA.

In summary, for the alternative, exemplary embodiments of Step 3:
a. Solvent exchange to ethyl acetate to enable removal of TMU by aqueous washes.
b. The control of the bis-amide by-product through a three-fold approach
   i. Discovery that the acid form of AB-007-5 is unstable and disproportionates forming the reactive di-acid form. Use of an alkaline metal salt form limits degradation.
   ii. A saturated brine wash of an organic solution of AB-007-5 in its acid form removes traces of di-acid.
   iii. The bis-amide impurity has a surprisingly low solubility in the reaction mixture prior to further work up and can be mostly removed by filtration of the reaction mixture.

In summary, these exemplary, alternative conditions in alternative embodiments ensure, e.g., good control of the rate of carbon dioxide gas evolution throughout the course of the reaction, along with isolation of the Step 2 product as a “free base” form to avoid toluene sulfonic acid carry over to the subsequent coupling step.

### Preparative Chromatography

In one embodiment, Scheme Ia conditions for the final step of coupling AB-007-4 with AB-007-5 to give the AB-007 API requires preparative chromatography to isolate the API free from some significant process impurities.

Using chromatography as a final preparative step for the preparation of material for clinical trials may introduce a steep ongoing cost burden for alternative, e.g., scaled up, manufacture of AB-007. This may be because chromatography removes related substances, giving (resulting in) a very pure API. If this very pure API is used to qualify through toxicology testing and clinical trials (e.g., if the very pure API is used in toxicology testing for FDA approval for clinical trials), it would make it very difficult to avoid using (not also use) chromatography in alternative, e.g., scaled up, protocols. Not using chromatography (or an alternative apparatus or protocol yielding a high quality purification) in alternative, e.g., scaled up, protocols would result in increased impurity levels; and these impurity levels would then exceed qualified (low impurity) levels present in the chromatographed material, and thus would not be acceptable to the FDA.

Removal of Impurities

In alternative embodiments, major impurities are removed. In Scheme Ia, two major impurities were identified:
(i) tetramethylurea by-product of coupling chemistry, and
(ii) symmetrical bis-amide formed from reaction of AB-007-4 with bis-acid impurity present in AB-007-5.

Tetramethylurea was successfully removed without need for chromatography by solvent exchange and aqueous washing of reaction product solution. It was found that bis-acid impurity present in AB-007-5 is due to disproportionation of AB-007-5 into (reactive) bis-acid and (unreactive) bis-ester, catalyzed by residual mineral acid present in AB-007-5. To address this, AB-007-5 raw material is prepared in neutral-pH sodium salt form instead of free acid form so as to avoid residual acidity causing its disproportionation.

In one embodiment, a brine wash also is employed to remove the small amount of remaining bis-acid in AB-007-5 and, based on solubility studies which showed low solubility of the unwanted bis-amide, the small amount of bis-amide formed in the final step coupling was removed by filtration and API recrystallization.

In these alternative embodiments, process changes allowed definition of a robustly scaleable and efficient manufacturing process giving high purity AB-007 API without need for expensive preparative chromatography.

In an alternative embodiment, the invention provides a “scaled up” process technology providing kilogram quantities of AB-007 suitable for clinical trials.

### Scheme II: An Exemplary Synthesis of AB-007-API

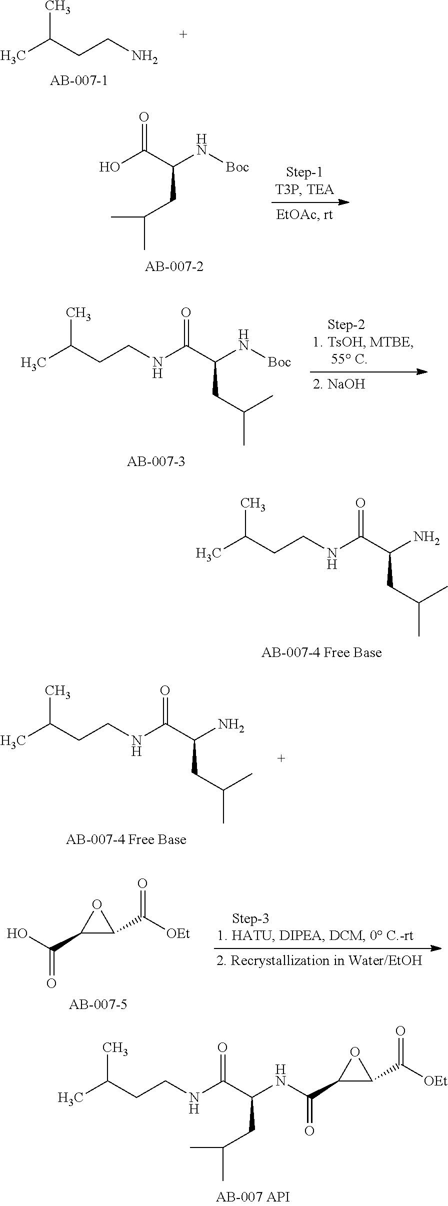

### Exemplary Schemes of the Invention for the Synthesis of AB-007-API

### Step 1: Preparation of AB-007-3

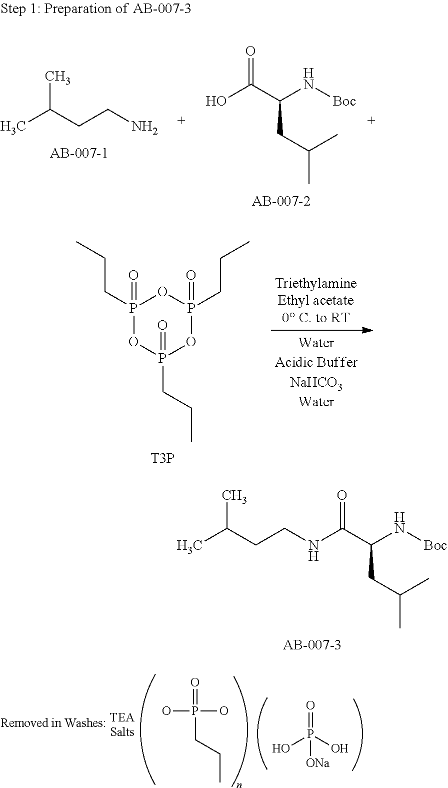

### Step 2: Preparation of AB-007-4 Free Base

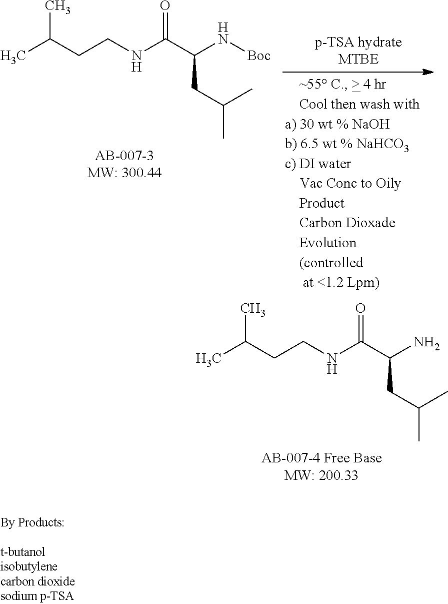 [SMILES: CC(C)CCNC(=O)[C@H](CC(C)C)NC(=O)OC(C)(C)C ; CC(C)CCNC(=O)[C@@H](N)CC(C)C]

### Step 3: Preparation of AB-007 API

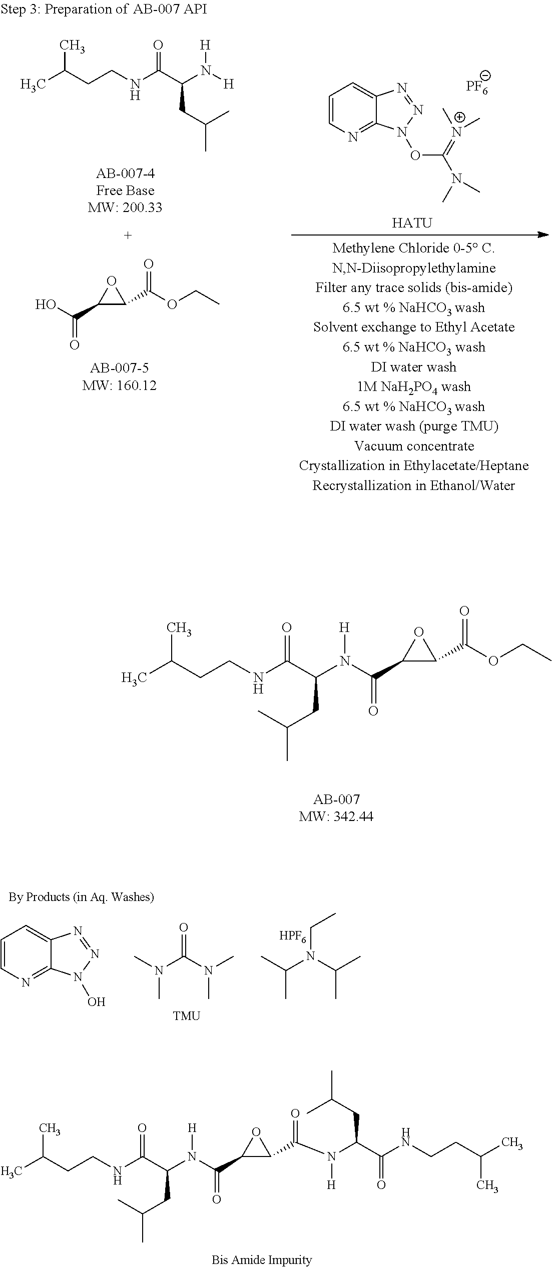

### Exemplary Protocols of the Invention

### Preparation of AB-007-3

AB-007-2 (2.152 kg, 1.0 eq.) was dissolved in ethyl acetate (2 L/kg) and heated to reflux with a Dean-Stark trap to collect the water which was removed by distillation. After the water was removed, the solution was cooled to <10° C. and triethylamine (5.26 kg, 6 eq.) was added while maintaining the temperature at <10° C. followed by AB-007-1 (0.762 kg, 1.0 eq.). The mixture was cooled to approximately 0° C. and T3P® (6.57 kg, 1.2 eq.) (50% wt. solution in ethyl acetate) was added slowly at 0-5° C. The reaction mixture was stirred for one hour and then warmed to ambient temperature for at least 4 hours. The mixture was cooled to 0° C. and quenched with water (10 vol.). The organic phase was washed twice with 1M pH 4 phosphate buffer solution and twice with 6.5% sodium bicarbonate solution.

After a final water wash (5 vol.), the organic phase was vacuum concentrated to a minimum stirring volume and then diluted with water (2.4 vol.). After the removal of the residual solvent by vacuum distillation, a soft stirrable pasty mixture was obtained. Acetone (3.8 kg, 1.2 vol.) was added and the mixture was warmed to 50° C. to give a clear solution. Cooling to 20-25° C. provided a granular slurry of AB-007-3. The product AB-007-3 was isolated by filtration and dried at 45° C. under vacuum. A total of 2.238 kg AB-007-3 was obtained.

Preparation of AB-007-4

AB-007-3 (2.328 kg, 1.0 eq.) was dissolved in MTBE (7 vol.) and was added slowly to a refluxing solution of p-TSA (1.769 kg, 1.2 eq.) in MTBE (5 vol.). The AB-007-3 solution addition rate was controlled to provide a safe rate of gas evolution (approximately 4 hrs total time at approximately 2 kg scale). After the addition was complete, reflux was continued until the reaction was complete by HPLC (approximately 2 to 3 hrs). The reaction mixture was cooled to ambient temperature and 30% sodium hydroxide solution (1.8 eq.) was added which precipitated the sodium p-TSA salt. The precipitated salts were dissolved with a minimum amount of water and the phases were separated. The rich MTBE phase was then washed with a small volume (approximately 3% total vol.) of 6.5% sodium carbonate solution and then a small volume (approximately 3% total) of water. The rich MTBE solution was vacuum concentrated at <50° C. to a light yellow oil. A total of 1.288 kg of AB-007-4 free base was obtained.

Preparation of AB-007 API

AB-007-5 (0.776 kg, 1.0 eq.) was dissolved in DCM (20 vol.) and the solution was cooled to 0-5° C. HATU (1.935 kg, 1.05 eq.) was charged to the cold mixture (no exotherm and no reaction). A solution of

AB-007-4 Free Base (0.973 kg, 1.0 eq.) in DIPEA (0.689 kg, 1.10 eq.) was added slowly while maintaining the reaction temperature at 0-5° C. The reaction was stirred for two hours at 0 to 5° C. and checked by HPLC. If reaction was not complete, the reaction mixture was warmed to 15 to 25° C. and stirred until the reaction was complete by HPLC. The reaction mixture was cooled to 0 to 5° C. and stirred for at least 30 minutes and then filtered to remove any visible solids. (The bis-amide impurity was fairly in-soluble in the reaction mixture and most of it could be removed at this point). The filtrate was washed with 6.5% sodium bicarbonate solution and vacuum concentrated and the solvent exchanged to ethyl acetate solution. The rich ethyl acetate solution was then washed with 6.5% sodium carbonate solution, twice with water, 1M phosphate buffer, 6.5% sodium carbonate solution, and water. The rich ethyl acetate solution was then vacuum concentrated with the addition of heptane to provide a product slurry. The slurry was filtered and the wet cake was vacuum dried at 45° C. to afford crude AB-007 API. At this stage, the bis-amide impurity was below 0.5% by HPLC. For the final recrystallization, the crude API was dissolved in ethanol (8 vol.) at 40 to 60° C. and the solution was polish filtered through an in-line filter and then water was added through an in-line filter. The mixture was heated to 55 to 65° C. to dissolve all solids and then cooled to 20-25° C. over approximately 4 hours. (Crystallization occurred at about 40 to 45° C.) The slurry was cooled to 5 to 10° C. and filtered. The wet cake was washed twice with cold ethanol/water (1/2, v/v, 1 vol.) and vacuum dried at 45° C. to a constant weight. A total of 1.096 kg of AB-007 API was obtained.

### Example III: Alternative Exemplary Protocols to Synthesize Deuterated Isoamylamine, E64d and E64c

Alternative exemplary synthetic schemes of the invention include exemplary protocols to synthesize deuterated isoamylamine, E64d and E64c, and in one embodiment, 11-deuterated (or eleven-deuterated, or 11-d) isoamylamine, E64d and E64c.

In one embodiment, an exemplary synthetic route for making isoamylamine-d11 (and equivalents) is (comprises):

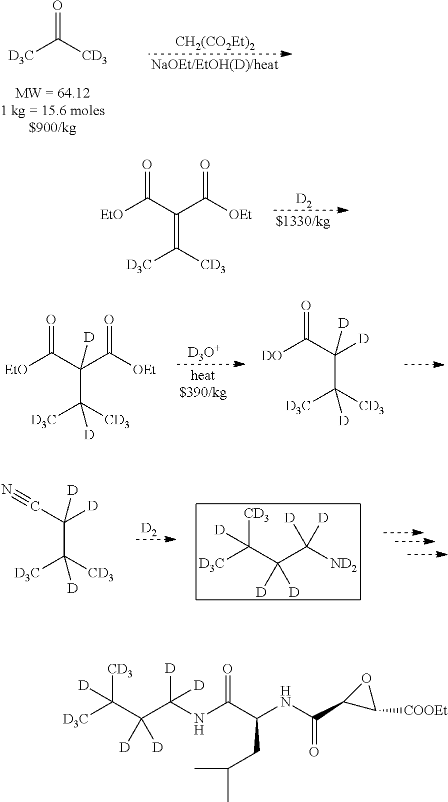

Estimated yield of isoamylamine-d11(13) on kg scale: 664 g of isoamylamine-d11(13) gives a projected amt of about 930 g E64D-d11
(5.11 moles amine synthesized if 80% yield is obtained on each of the five steps)
Estimated cost on kg scale of reagents: $2620+non-deuterated reagents/solvents
Comparison of deuterated vs. non-deuterated routes:
cost of d11 isoamylamine: $2620/kg
# steps to synthesize d11 isoamylamine: 5 (adds additional manufacturing costs as well)
cost of non-deuterated isoamyl amine: $82/kg (from Aldrich)
# steps to synthesize non-deuterated isoamylamine: Zero

In one embodiment, an exemplary synthetic route for making 11-deuterated (eleven deuterated) AB-007 (and equivalents) is (comprises) the following steps:

Steps 1 to 3 for the Preparation of AB-007 API (d-11)

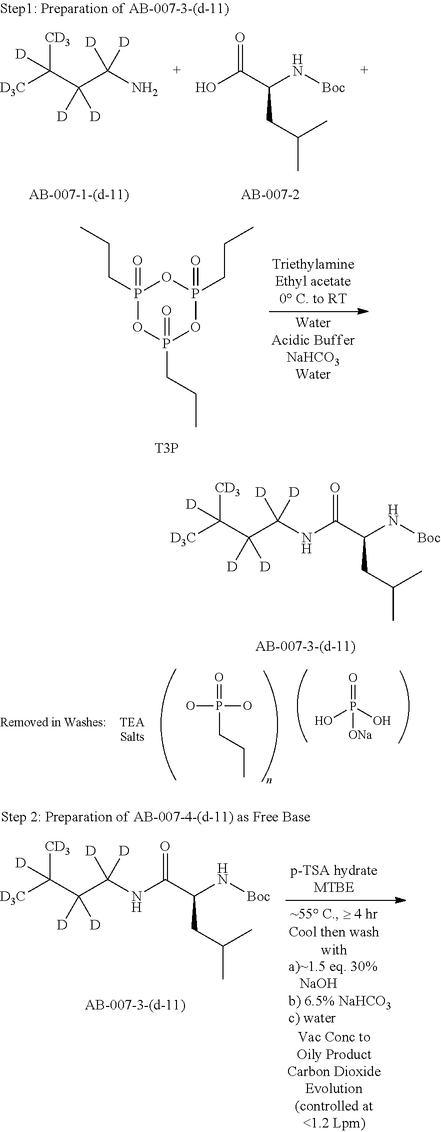

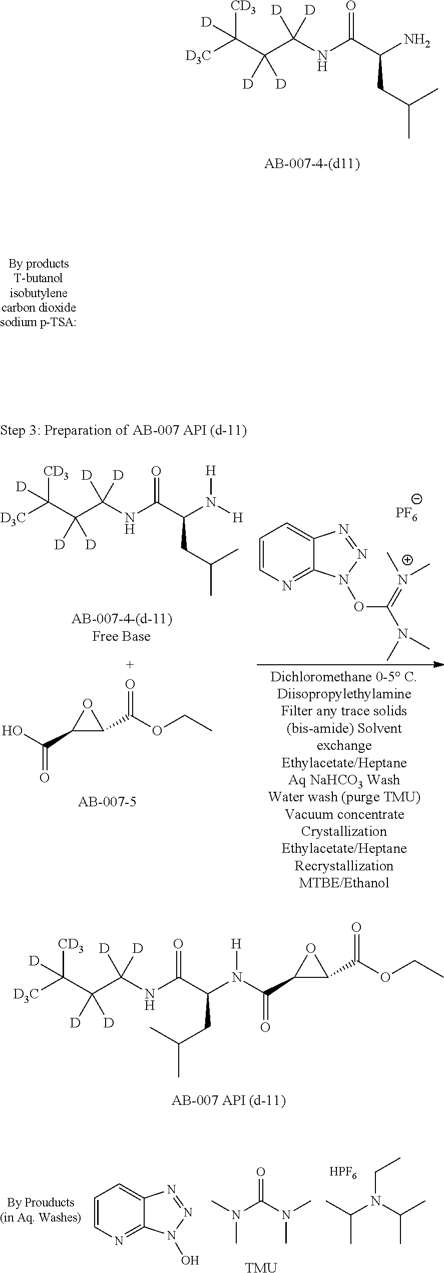

In one embodiment, the invention provides a method for synthesizing AB-007-3-(eleven deuterated, or d-11), comprising:

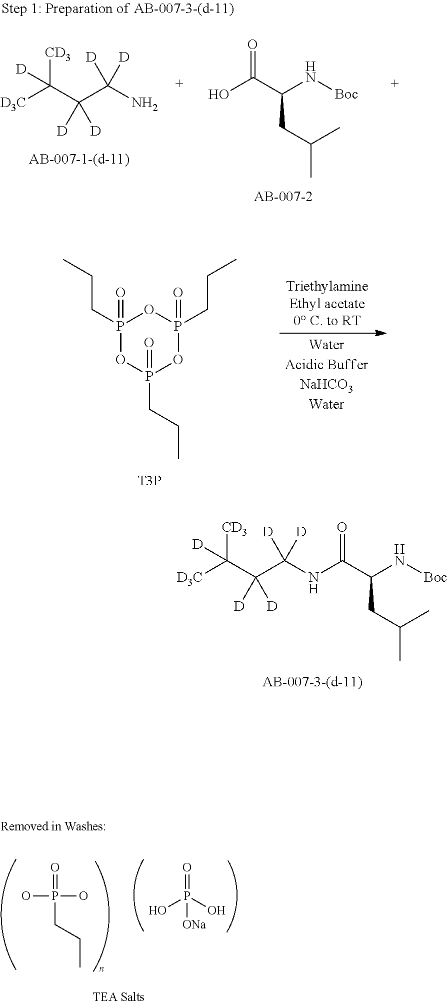

In one embodiment, the invention provides a method for synthesizing AB-007-3-(eleven deuterated, or d-11), comprising:

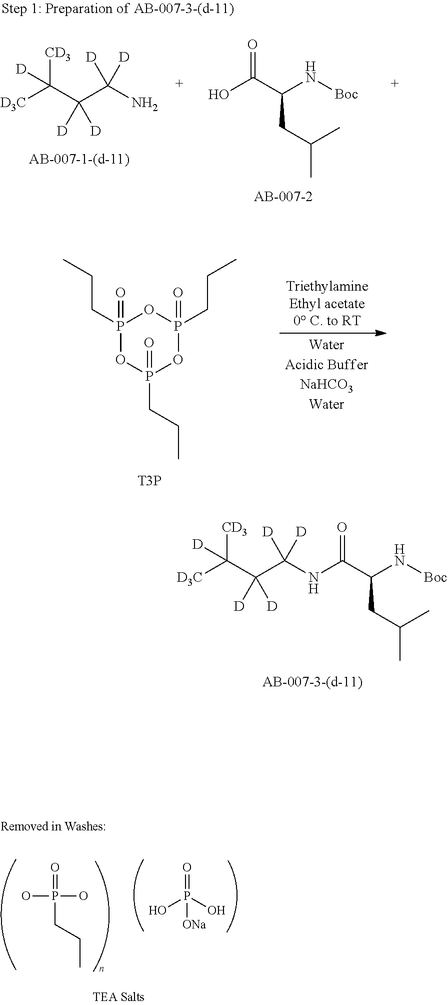

In one embodiment, the invention provides a method for synthesizing AB-007-4-(eleven deuterated, or d-11), comprising:

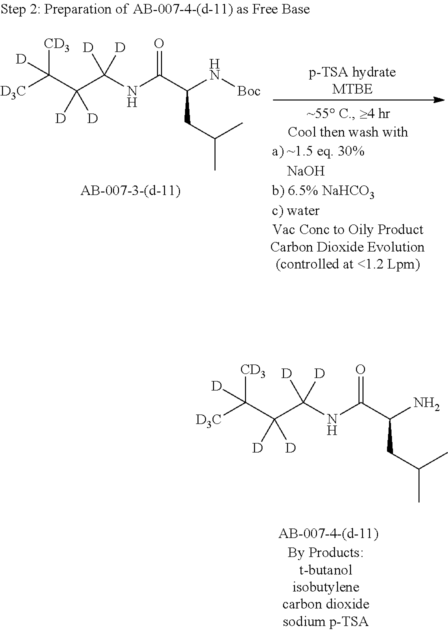 [SMILES: [2H]C([2H])([2H])C([2H])(C([2H])([2H])[2H])C([2H])([2H])C([2H])([2H])NC(=O)[C@H](CC(C)C)NC(=O)OC(C)(C)C ; [2H]C([2H])([2H])C([2H])(C([2H])([2H])[2H])C([2H])([2H])C([2H])([2H])NC(=O)[C@@H](N)CC(C)C]

In one embodiment, the invention provides a method for synthesizing AB-007-(eleven deuterated, or d-11), from AB-007-4-(eleven deuterated, or d-11) comprising:

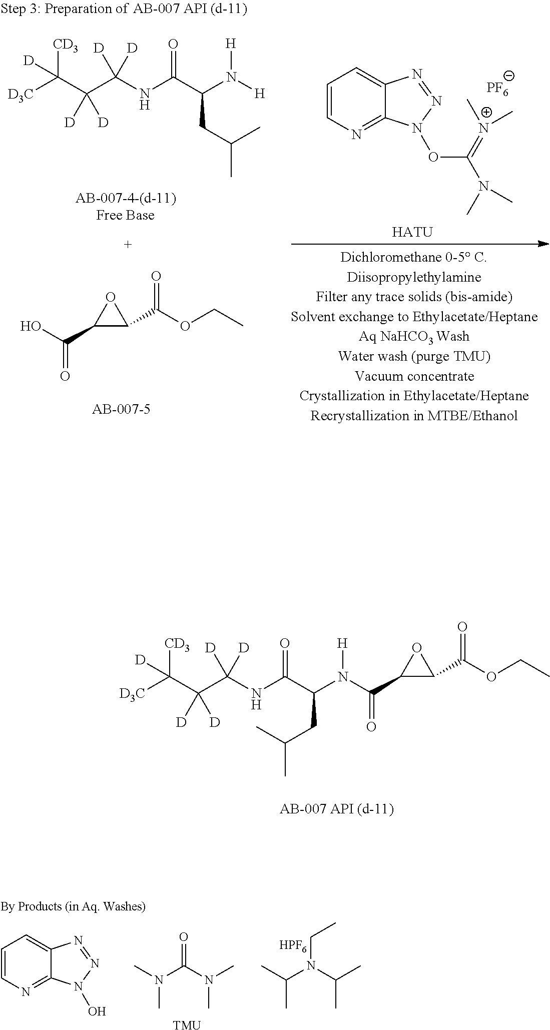

Although the foregoing invention has been described in some detail by way of illustration and example for purposes of clarity of understanding, it will be readily apparent to one of ordinary skill in the art in light of the teachings of this application that certain changes and modifications may be made thereto without departing from the spirit or scope of the appended claims. A number of aspects of the invention have been described. Nevertheless, it will be understood that various modifications may be made without departing from the spirit and scope of the invention. Accordingly, other aspects are within the scope of the following claims.

1: A composition comprising: a tosylate salt of AB-007-4; or, a tosylate salt of L-leucine isoamylamine; or, equivalents thereof.

2: A tosylate salt of AB-007-4 or a tosylate salt of L-leucine isoamylamine, or equivalents thereof.

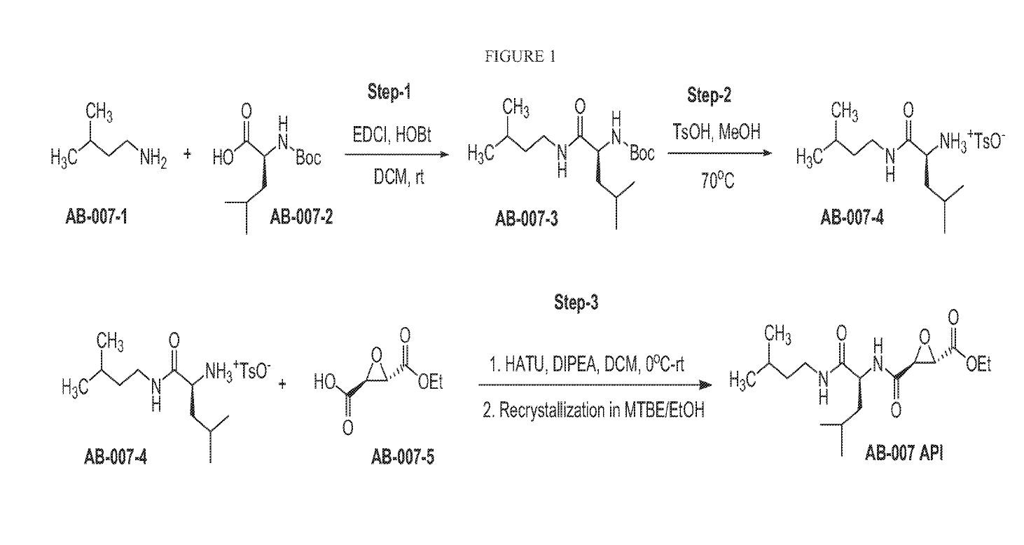

3: A synthetic scheme comprising at least one scheme: (a) as set forth in FIG. 1; (b) as set forth in Scheme Ia, Scheme Ib, Scheme II, Scheme C and/or Scheme D, of Example I; or (c) equivalents of (a) and (b).

4: (Original I): A synthetic scheme for making AB-007, comprising:
(a) Step 1 of the following Scheme, and equivalents thereof: 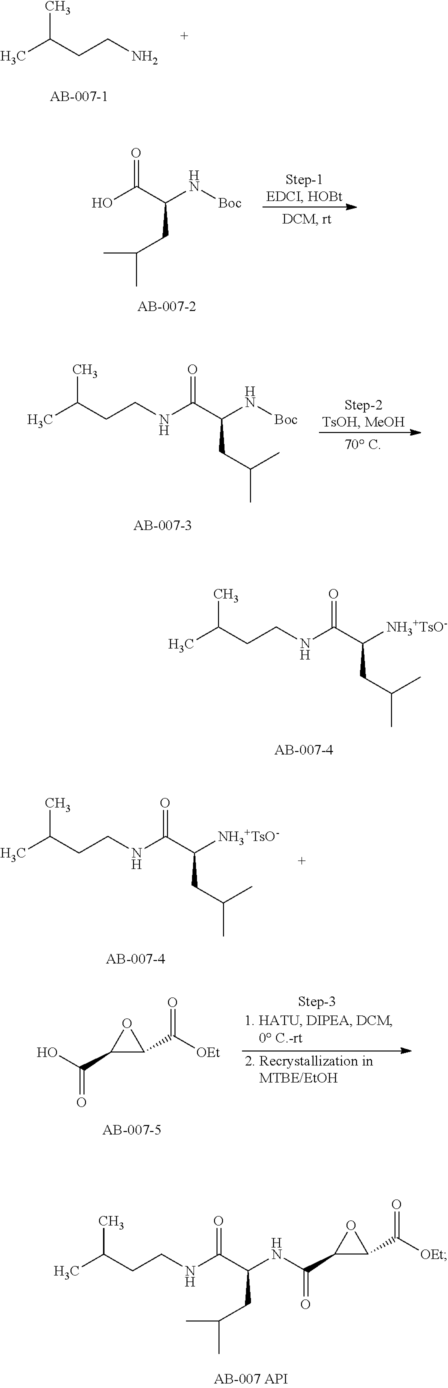
or
(b) the synthetic scheme of (a), wherein the AB-007 has one, two or all three of the three chiral centers of E64c.

5: A synthetic scheme comprising Step 2 of the following Scheme, and equivalents thereof:

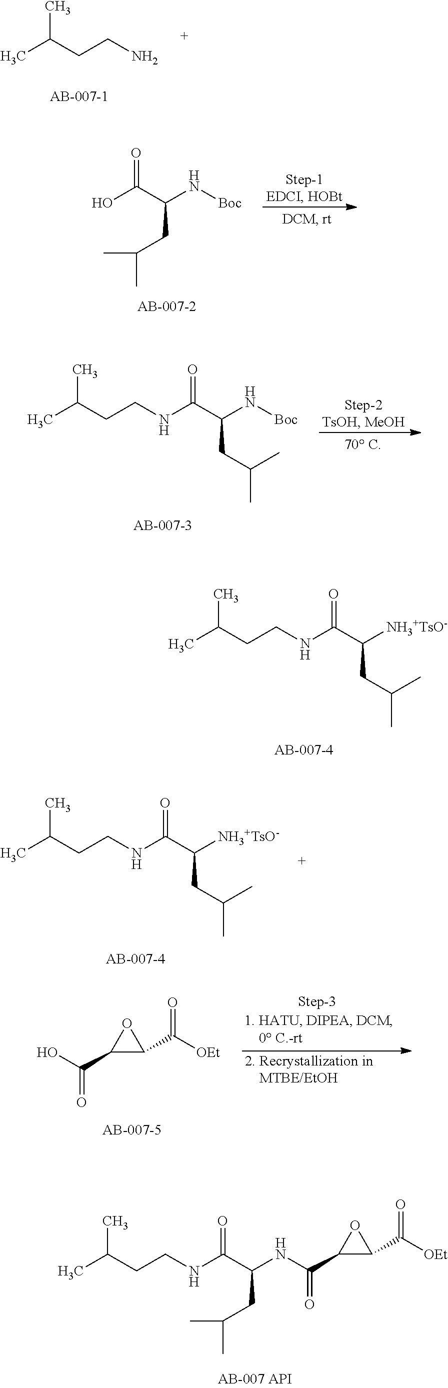

6: A synthetic scheme comprising Step 3 of the following Scheme, and equivalents thereof:

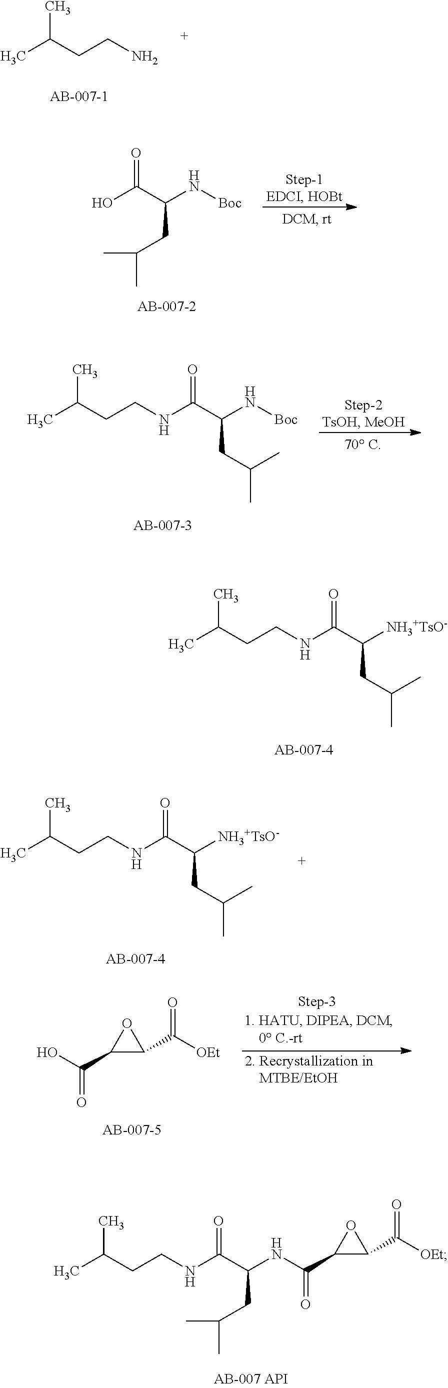

7: A synthetic scheme comprising Step 1 and Step 2 of the following Scheme, and equivalents thereof:

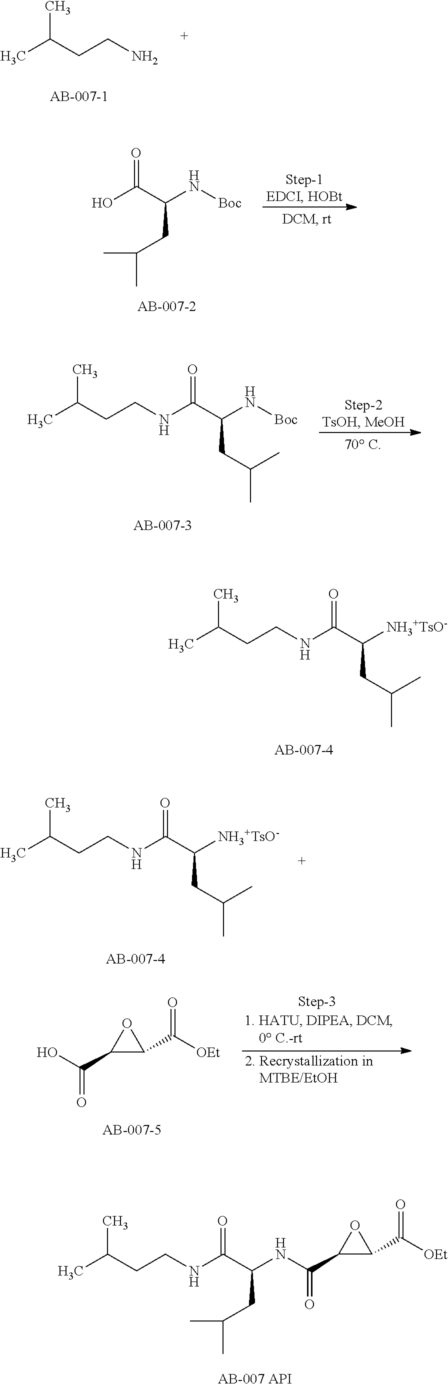

8: A synthetic scheme comprising Step 2 and Step 3 of the following Scheme, and equivalents thereof:

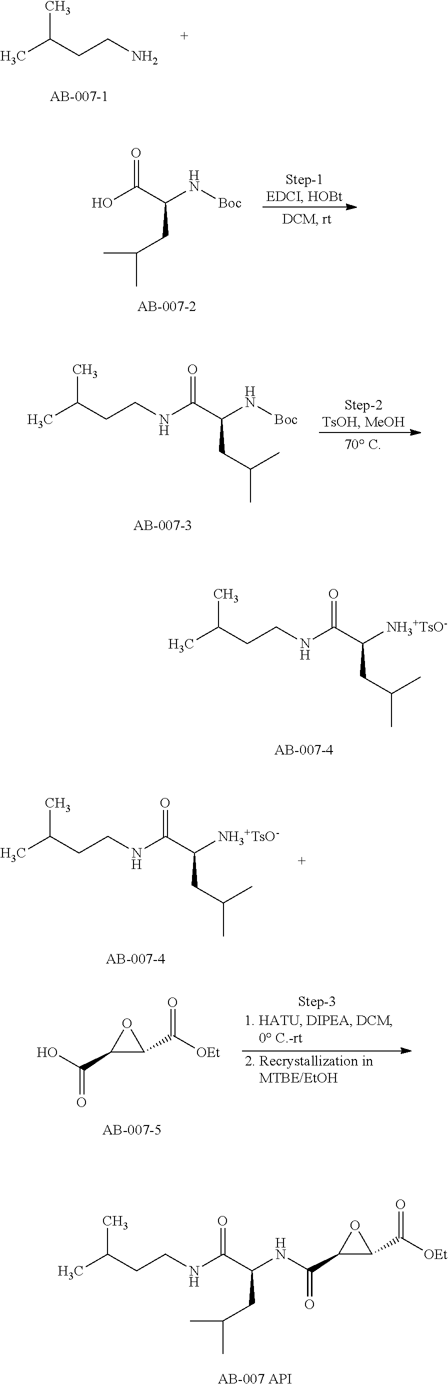

9: The synthetic scheme of claim 4, wherein:
(a) in place of using EDCI/HOBt standard peptide coupling conditions for a coupling reaction, optionally, as in Scheme I, a reagent comprising or consisting of propylphosphonic anhydride, or propane phosphonic acid anhydride (T3P®), is used for the coupling reaction;
(b) in place of using BOC deprotection of AB-007-2 starting material, optionally, before step 1 of Scheme I, alternative conditions comprising controlled addition of a solution of AB-007-3 into a warm solution of excess p-toluene sulfonic acid can be used to provide effective control of gas evolution rate by adjusting the addition rate of the AB-007-3 solution, as needed; these conditions can be used to ensure a good control of the rate of carbon dioxide gas evolution throughout the course of the reaction, along with isolation of the Step 2 product as a “free base” form to avoid toluene sulfonic acid carry over to the subsequent coupling step;
(c) drying of a hydrated AB-007-2 starting material, optionally, before step 1 of Scheme I, and optionally removal of water of a hydrated AB-007-2 by azeotropic drying;
(d) isolating a solid AB-007-3, optionally, after step 1 of Scheme I, by crystallization, and optionally by crystallization from water and acetone.
(e) for a BOC deprotection step optionally, step 2 of Scheme I, controlling addition of an AB-007-3 solution into an excess warm acid solution for control of gas evolution through addition control;
(f) isolating AB-007-4, optionally, after step 2 of Scheme I, as a free base form to avoid issues with carry-over of residual p-TSA;
(g) exchanging solvent to ethyl acetate in step 3, Scheme I, to enable removal of TMU (or 1,1,3,3-tetramethylurea) by one or more aqueous washes;
(h) controlling bis-amide by-product in step 3, Scheme I, using an approach comprising:
(1) using an alkaline metal salt form to limit degradation of the acid form of AB-007-5, which can be unstable, causing disproportionates forming a reactive di-acid form;
(2) using saturated brine wash of an organic solution of AB-007-5 in its acid form to remove traces of di-acid; and/or
(3) removing a bis-amide impurity by filtration of the reaction mixture;
(i) in place of needing chromatography, two critical process impurities are controlled by process conditions comprising: for the control of tetramethylurea (TMU), after a solvent exchange to ethylacetate, an organic solution is washed with 6.5% (w/w) sodium bicarbonate solution, de-ionized water, and 1 M monosodium phosphate, and de-ionized water;
(j) in place of needing chromatography by solvent exchange and aqueous washing of reaction product solution to remove impurities, AB-007-5 raw material or AB-007-5 made by step 3, Scheme Ia, is prepared in a neutral-pH sodium salt form instead of a free acid form so as to avoid residual acidity causing its disproportionation;
(k) the method of (j), wherein a brine wash also is employed to remove small amounts of remaining bis-acid in AB-007-5; and optionally, small amount of bis-amide formed in the final step coupling is removed by filtration and AB-007 API recrystallization;
(l) the amount of bis-amide by-product can be controlled by a protocol comprising:
(1) using an alkaline metal salt form to limit degradation, and/or
(2) using a saturated brine wash of an organic solution of AB-007-5 in its acid form to remove traces of di-acid, and/or
(3) removing by filtration bis-amide impurities; or
(l) any combination of, or at least one of, (a) to (k), or all of (a) to (k).

10: A synthetic route for making isoamylamine-d11 and equivalents, or for making 11-deuterated AB-007 (or E64d) comprising the following steps:

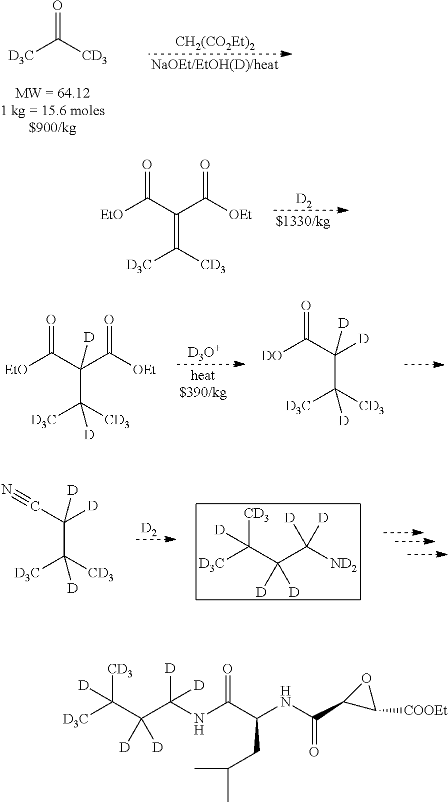

11: A method for synthesizing AB-007-3-(eleven deuterated, or d-11), or equivalents, comprising:

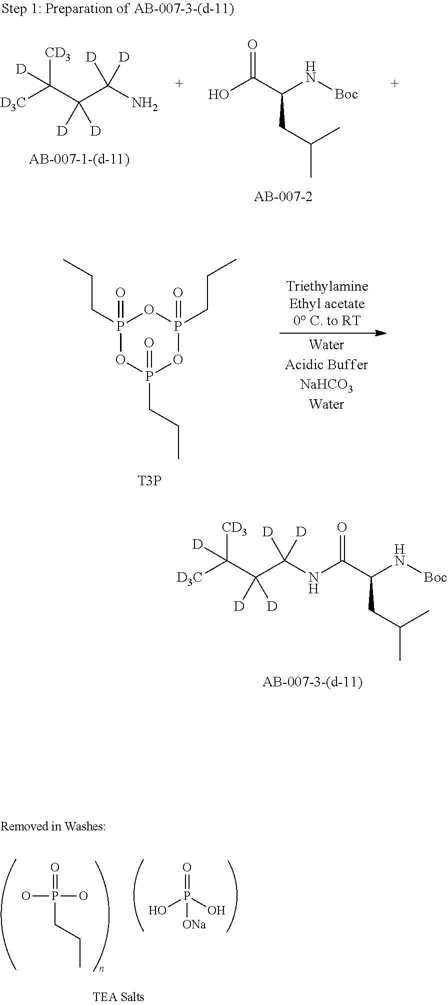

12: A method for synthesizing AB-007-4-(eleven deuterated, or d-11), or equivalents, comprising:

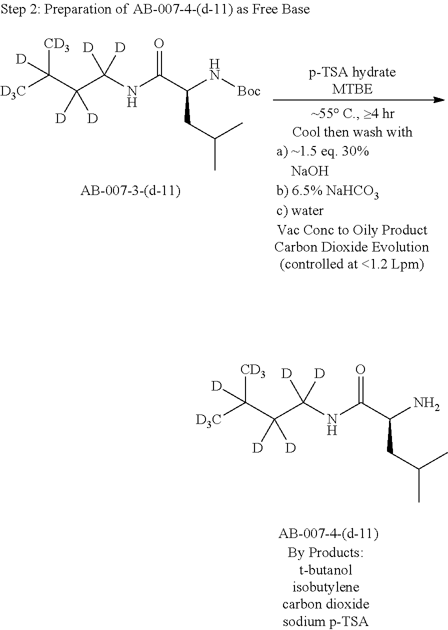 [SMILES: [2H]C([2H])([2H])C([2H])(C([2H])([2H])[2H])C([2H])([2H])C([2H])([2H])NC(=O)[C@H](CC(C)C)NC(=O)OC(C)(C)C ; [2H]C([2H])([2H])C([2H])(C([2H])([2H])[2H])C([2H])([2H])C([2H])([2H])NC(=O)[C@@H](N)CC(C)C]

13: A method for synthesizing AB-007-(eleven deuterated, or d-11), from AB-007-4-(eleven deuterated, or d-11), or equivalents, comprising: [chemical structure]
